(19) United States
(12) Patent Application Publication
(10) Pub. No.: US 2022/0202756 A1
(43) Pub. Date: Jun. 30, 2022

(54) GENETIC VARIANTS ASSOCIATED WITH RESPONSE TO TREATMENT OF NEUROLOGICAL DISORDERS

(71) Applicant: VISTAGEN THERAPEUTICS, INC., South San Francisco, CA (US)

(72) Inventor: Ralph H. SNODGRASS, South San Francisco, CA (US)

(21) Appl. No.: 17/601,464

(22) PCT Filed: Apr. 9, 2020
(86) PCT No.: PCT/US20/27440

§ 371 Date: Oct. 5, 2021

Related U.S. Application Data
(60) Provisional application No. 62/831,417, filed on Apr. 9, 2019.
(60) Provisional application No. 62/878,433, filed on Jul. 25, 2019.

(51) Int. Cl. A61K 31/198; A61P 25/24; C12Q 1/6883

(52) U.S. Cl. CPC A61K 31/198; A61P 25/24; C12Q 2600/156; C12Q 2600/106; C12Q 1/6883

(57) ABSTRACT

Method of treating depression, major depressive disorder, suicidal ideation and related disorders are described by administering L-4-chlorokynurenine (L-4-CI-KYN) to a subject based on their genotype.

### CROSS REFERENCE TO RELATED APPLICATIONS

This application claims priority to U.S. application 62/831,417 filed Apr. 9, 2019 and to U.S. application 62/878,433 filed Jul. 25, 2019; the disclosures of which are incorporated herein by reference.

### FIELD OF THE INVENTION

The invention relates to methods of treating neurological disorders, including depression, major depressive disorder, suicidal ideation, and other disorders mediated at least in part by the NMDA receptor, by administering L-4-chlorokynurenine (L-4-CI-KYN) to a subject based on the subject's genotype.

### BACKGROUND

Depression includes common but serious disorders of the brain characterized by combinations of signs and symptoms that may include feelings of hopelessness, guilt, worthlessness, and/or sadness alongside changes in sleep and/or eating patterns.

While most people will experience depressed mood at some point during their lifetime, major depressive disorder (MDD) is different. MDD is the chronic, pervasive feeling of utter unhappiness and suffering, which impairs daily functioning. Symptoms of MDD include diminished pleasure in activities, changes in appetite that result in weight changes, insomnia or oversleeping, psychomotor agitation, loss of energy or increased fatigue, feelings of worthlessness or inappropriate guilt, difficulty thinking, concentrating or making decisions, and thoughts of death or suicidal and attempts at suicidal. MDD is the most prevalent type of depressive disorder.

Risk for suicide is significantly increased in depressive disorders, but subjects may respond differentially to medication versus depressive symptoms as a whole. Suicide, also known as completed suicide, is the “act of taking one's own life”. Attempted suicide or non-fatal suicidal behavior is self-injury with the desire to end one's life that does not result in death. Suicidal ideation is the medical term for thoughts about or an unusual preoccupation with suicidal, or thoughts of ending one's life or not wanting to live anymore but not necessarily taking any active efforts to do so. When suicidal ideation occurs, it is often accompanied by feelings of worthlessness or inappropriate guilt, as well as recurrent thoughts of death or suicidal ideation and guilt is an accepted proxy for suicidal. Suicidal ideation is generally associated with depression (at about 60-70% of all cases).

The range of suicidal ideation varies greatly from fleeting to chronic and progress to detailed planning, role playing, and unsuccessful attempts, which may be deliberately constructed to fail or be discovered or may be fully intended to result in death. Although not all who have suicidal ideation go on to make suicide attempts, a significant proportion do.

U.S. Pat. No. 9,993,453 describes methods of treating depression comprising the step of orally administering to a human subject in need thereof a therapeutically effective amount of L-4-CI-KYN, a prodrug of 7-chlorokynurenic acid (7-CI-KYNA), which is a synthetic, chlorinated analogue of an endogenous neuromodulator, kynurenic acid. As described in Zanos et al., J Pharmacol. Exp. Ther. 355:76-85, (2015), 7-chlorokynurenic acid has been shown to prevent excitotoxic and ischemic neuronal damage, but like most GlyB antagonists does not cross the blood-brain barrier. Thus, its clinical use is limited. In contrast, however, L-4-CI-KYN readily gains access to the central nervous system (CNS) after administration. L-4-CI-KYN is efficiently converted to 7-chlorokynurenic acid within activated astrocytes, and brain levels of 7-chlorokynurenic acid are increased at sites of neuronal injury or excitotoxic insult as a result of astrocyte activation.

### SUMMARY OF THE INVENTION

The invention generally relates to a method of treating a subject with neurological disorders, including depression, major depressive disorder, and/or suicidal ideation, by administering L-4-CI-KYN to the subject based on the subject's genotype.

In one aspect, the invention relates to a method of predicting responsiveness to treatment with L-4-CI-KYN by detecting at least one of a kynurenine 3-monooxygenase (KMO) gene variant, a SLC7A5 neutral amino acid transporter gene variant, a brain derived neurotrophic factor (BDNF) gene variant, and an aminocarboxymuconate semialdehyde decarboxylase (ACMSD) gene variant, in a subject suffering from depression, major depressive disorder, and/or suicidal ideation, where the method includes obtaining a sample from the subject; and assaying the sample to detect one or more of a T allele of the single nucleotide polymorphism (SNP) rs61825638 in KMO, a T allele of the SNP rs28582913 in SLC7A5, the common G allele at the location of rs6265 in BDNF, and a C allele of the SNP rs2121337 in ACMSD.

In another aspect, the invention relates to a method for the treatment of depression, major depressive disorder, and/or suicidal ideation in a subject, where the method includes obtaining a sample from the subject; and assaying the sample to detect one or more of a T allele of the single nucleotide polymorphism (SNP) rs61825638 in KMO, a T allele of the SNP rs28582913 in SLC7A5, an A allele of the SNP rs6265 in BDNF, and a C allele of the SNP rs2121337 in ACMSD, and administering a therapeutically effective amount of L-4-CI-KYN to the subject to treat the depression, major depressive disorder, or suicidal ideation in the subject, based on the presence of at least one of these detected variants in the sample.

Yet another aspect of the invention relates to a method of predicting the potential responsiveness of a subject suffering from depression, major depressive disorder, and/or suicidal ideation together with treatment with L-4-CI-KYN administered to the subject, where the method includes obtaining a sample from the subject and assaying the sample to detect one or more of a T allele of the single nucleotide polymorphism (SNP) rs61825638 in KMO, a T allele of the SNP rs28582913 in SLC7A5, a A allele of the SNP rs6265 in BDNF, and a C allele of the SNP rs2121337 in ACMSD, and where the absence or presence of at least one of the gene variants is indicative of the subject's responsiveness to the treatment.

### BRIEF DESCRIPTION OF THE FIGURES

FIG. 1 shows the effect of a SLC7A5 gene variant on L-4-chlorokynurenine transport across the blood-brain barrier. The LAT1 (SLC7A5) protein actively transports AV-101 (L-4-chlorokynurenine), and other neutral amino acids, from the blood, across the blood brain barrier, into the brain. In humans, LAT1 exists in multiple genetic variations. The genetic LAT1 variant containing the rs28582913 C/T variation results in a more active transport of AV-101 into the brain compared to its most common genetic form. This enables the local production in the brain of relatively higher levels of 7-chlorokynurenic acid, the active metabolite of L-4-chlorokynurenine (AV-101), in the brain of patients with the rs28582913 C/T variation.

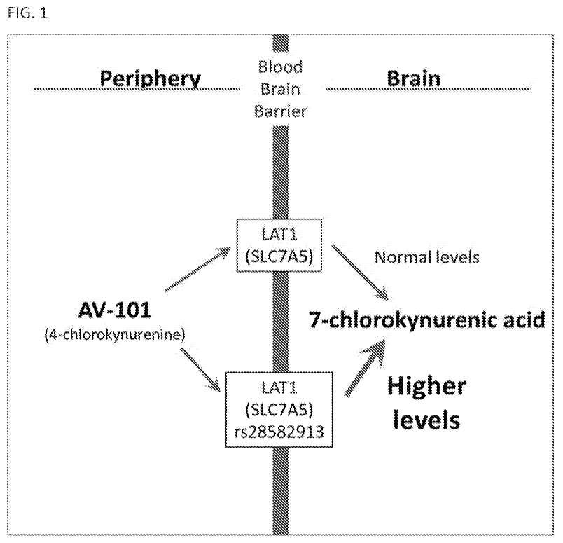

FIG. 2 shows the effect of a KMO gene variant on brain levels of 7-chlorokynurenic acid. In the brain, AV-101 has two major pathways of metabolism. One involves the kynurenine amino transferase (KAT) enzyme which produces 7-chlorokynurenic acid, the therapeutically active molecule of AV-101. The other pathway involves KMO enzyme that results in the degradation of AV-101. The more active the KMO degradative pathway is, the less AV-101 there is available for conversion to the active 7-chlorokynurenic acid metabolite. In humans, there are genetic variations in the KMO gene that affect its functional activity. The rs61825638 CIT variation results in a relatively less functional KMO, and therefore results in more 7-chlorokynurenic acid.

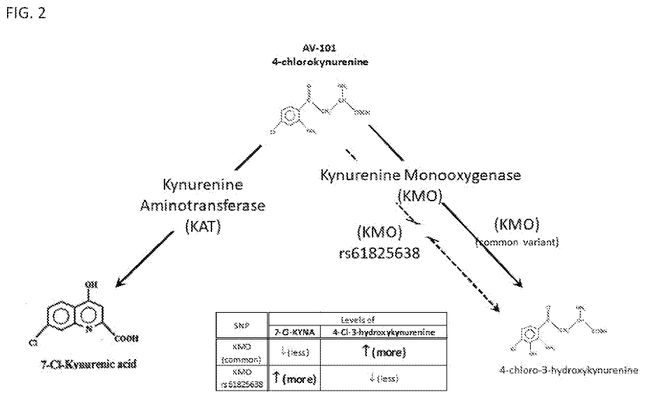

FIG. 3 shows a simplified diagram of the kynurenine pathway. ACMSD, amino-β-carboxymuconate-semialdehyde-decarboxylase; HAAO, hydroxyanthranilate 3,4-dioxygenase; IDO, indoleamine 2,3-dioxygenases; KAT, kynurenine aminotransferases; KMO, kynurenine 3-monooxygenases; KYNU, kynureninase; NAD, nicotinamide adenine dinucleotide; APRT, quinolinate phosphoribosyltransferase; TDO, tryptophan 2,3-dioxygenase. This figure also includes a description of the functional association of the rs2121337 (T→C) SNP in the ACMSD gene. This polymorphism leads to a reduction of ACMSD activity, and a concomitant increase of quinolinic acid levels, and is associated with higher rates of suicide attempts.

### DETAILED DESCRIPTION

The invention is described below, with reference to detailed illustrative embodiments. It will be apparent to persons skilled in the art that the invention may be embodied in a wide variety of forms, some of which may be quite different from those of the disclosed embodiments. Consequently, the specific structural and functional details disclosed below are merely representative and do not limit the scope of the invention.

It is believed that a subject's clinical response to L-4-CI-KYN is in part determined by subject genotypes relating to a subject's metabolism of L-4-CI-KYN and also to the transport of L-4-CI-KYN across the subject's blood-brain barrier.

KMO is a flavin adenine dinucleotide (FAD)-dependent monooxygenase and is located in the outer mitochondrial membrane where it converts L-kynurenine to 3-hydroxykynurenine. Perturbations in the levels of kynurenine pathway metabolites have been linked to the pathogenesis of a spectrum of brain disorders (Schwarcz, R., Bruno, J. P., Muchowski, P. J. & Wu, H.-Q. Kynurenines in the mammalian brain: when physiology meets pathology. Nature Rev. Neurosci. 13, 465-477 (2012), as well as cancer (Platten, M., Litzenburger, U. & Wick, W. The aryl hydrocarbon receptor in tumor immunity. Oncoimmunology 1, 396-397 (2012), and Liu, X., Newton, R. C., Friedman, S. M. & Scherle, P. A. Indoleamine 2,3-dioxygenase, an emerging target for anti-cancer therapy. Curr. Cancer Drug Targets 9, 938-952 (2009)), and several peripheral inflammatory conditions (Filippini, P. et al. Emerging concepts on inhibitors of indoleamine 2,3-dioxygenase in rheumatic diseases. Curr. Med. Chem. 19, 5381-5393 (2012). Marta Amaral et al., Nature 496, 382-385 (2013). The KMO locus with Reference SNP cluster ID rs61825638 has a single nucleotide polymorphism, wherein the minor allele, T instead of C, at position 241550102 on chromosome 1, is present in approximately 3-30% of the population depending on ethnicity (https://www.ncbi.nlm.nih.gov/snp/rs61825638#frequency_tab), and results in a decrease of KMO synthesis and a concomitant 2.14-fold increase in KYNA production. (Long, et al., 2017, Nature Genetics, 49:568-581.)

Without being bound to any particular theory, it is believed that the T allele of the rs61825638 SNP is associated with an increase in responsiveness to L-4-CI-KYN due to two effects: 1) KMO represents an alternate pathway away from KAT and the production of 7-CI-KYNA; therefore, a reduction of KMO activity in the brain would make more of the L-4-CI-KYN administered to a subject available and increase the conversion to the active metabolite, 7-CI-KYNA, in the brain; 2) KMO is highly expressed in the PBMCs, liver, spleen, and kidneys, and reduced activity in the periphery would make more L-4-CI-KYN administered to a subject available for transport into the brain.

The L-Type Amino Acid Transporter 1 (LAT1, SLC7A5) is part of the SLC7 family and forms a heterodimer with CD98 via a disulphide bond (Mastroberardino, L. et al. Amino-acid transport by heterodimers of 4F2hc/CD98 and members of a permease family. Nature 395, 288-291, doi: 10.1038/26246 (1998)). CD98 (4F2hc, SLC3A2) is a type II glycoprotein that functions as a chaperone for LAT1, stabilizing and facilitating its translocation to the plasma membrane. LAT1 is the functional unit of the complex (Napolitano, L. et al. LAT1 is the transport competent unit of the LAT1/CD98 heterodimeric amino acid transporter. The international journal of biochemistry & cell biology, doi: 10.1016/j.biocel.2015.08.004 (2015)) and substrates include a range of large neutral amino acids such as tyrosine, leucine, isoleucine, valine and phenylalanine as well as pharmaceutical drugs including L-DOPA and gabapentin. (Dickens, D. et al. Transport of gabapentin by LAT1 (SLC7A5). Biochem Pharmacol 85, 1672-1683, doi: 10.1016/j.bcp.2013.03.022 (2013); Uchino, H. et al. Transport of amino acid-related compounds mediated by L-type amino acid transporter 1 (LAT1): insights into the mechanisms of substrate recognition. Molecular pharmacology 61, 729-737 (2002)). The SLC7A5 locus with rsid rs28582913 has a single nucleotide polymorphism, wherein the minor allele, T instead of C, at position 87843494 on chromosome 16, is present in approximately 18-56% of the population depending on ethnicity (https://www.ncbi.nlm.nih.gov/snp/rs28582913#frequency_tab), and results in a 1.94 fold increase in the kynurenine metabolite. (Long, et al., 2017, Nature Genetics, 49:568-581.) AV-101 is a substrate for the LAT1 transporter. HEK-293 cells overexpressing LAT1 show a substantial increase in AV-101 uptake. AV-101 uptake is substantially reduced, when JPH203, a LAT1 inhibitor is added. Human brain endothelial cells also show significant uptake of AV-101. In the presence of JPH203, this uptake is significantly diminished. Additionally, physiological amino acids reduce LAT1 mediated transport of AV-101.

Without being bound to any particular theory, it is believed that the T allele of the rs28582913 SNP would be associated with an increase in responsiveness to L-4-CI-KYN due to an increase in the active transport of L-4-CI-KYN into the brain, resulting in the production of more 7-CI-kynureninic acid, the active metabolite.

The upregulation of BDNF has been associated with positive therapeutic effect of fast acting antidepressants. See Kato, T., et al. (2017). “BDNF release and signaling are required for the antidepressant actions of GLYX-13.” Mol Psychiatry; Lepack, A. E., et al. (2016). “Fast-acting antidepressants rapidly stimulate ERK signaling and BDNF release in primary neuronal cultures.” Neuropharmacology 111: 242-252; Lepack, A. E., et al. (2014). “BDNF release is required for the behavioral actions of ketamine.” Int J Neuropsychopharmacol 18(1): 10.1093/ijnp/pyu1033 pyu1033; Quesseveur, G., et al. (2013). “BDNF overexpression in mouse hippocampal astrocytes promotes local neurogenesis and elicits anxiolytic-like activities.” Transl Psychiatry 3: e253; Li, C. F., et al. (2016). “Activation of hippocampal BDNF signaling is involved in the antidepressant-like effect of the NMDA receptor antagonist 7-chlorokynurenic acid.” Brain Res 1630: 73-82; Gerhard, D. M. and R. S. Duman (2018). “Rapid-Acting Antidepressants: Mechanistic Insights and Future Directions.” Curr Behav Neurosci Rep 5(1): 36-47; Haile, C. N., et al. (2014). “Plasma brain derived neurotrophic factor (BDNF) and response to ketamine in treatment-resistant depression.” Int J Neuropsychopharmacol 17(2): 331-336; Laje, G., et al. (2012). “Brain-derived neurotrophic factor Va166Met polymorphism and antidepressant efficacy of ketamine in depressed patients.” Biot Psychiatry 72(11): e27-28.). BDNF, rs6265 (Va166Met, G→A SNP) has been shown to reduce intracellular trafficking and activity-dependent secretion of BDNF (Egan, M. F., et al. (2003). “The BDNF va166met polymorphism affects activity-dependent secretion of BDNF and human memory and hippocampal function.” Cell 112(2): 257-269.) BDNF, rs6265 (Va166Met, G→A SNP) is associated with a lower response to ketamine in patients experiencing an MDD episode, compared to the Val/Val allele. (Laje, G., et al. (2012)). BDNF is implicated in the pathophysiology of MDD and suicide, and subjects with the Met allele have an increased risk of depression. Youssef, M., et al., “Association of BDNF Va166Met Polymorphism and Brain BDNF Levels with Major Depression and Suicide”, Int J Neuropsychopharmacol (2018) 21(6): 528-538. It is believed that the A allele of rs6265 would be associated with a decrease in responsiveness to L-4-CI-KYN.

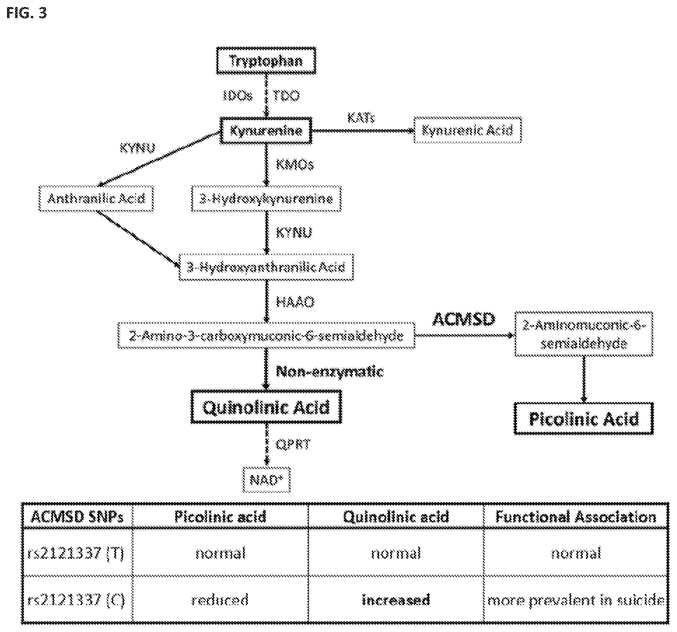

Increased levels of quinolinic acid (QA), a neurological excitotoxin, is associated with various neurological disorders (Brundin, L., et al. (2016). “An enzyme in the kynurenine pathway that governs vulnerability to suicidal behavior by regulating excitotoxicity and neuroinflammation.” Transl Psychiatry 6(8): e865). QA is a late appearing metabolite produced from the metabolism of tryptophan (see FIG. 3). Instead of conversion to QA, the precursor to QA can be metabolized by the ACMSD enzyme into a “beneficial” non-toxic metabolite, picolinic acid (PIC). Therefore, it is believed that a mutation that results in the reduced activity of ACMSD results in higher levels of the toxic QA. The minor C allele of SNP rs2121337 is such a mutation. Besides being more prevalent in suicidal patients, rs2121337 (T→C) is also associated with a higher QA/PIC ratio in the CSF. Brundin, L., et al. (2016). Out of seven SNPs, only rs2121337 showed significant association with suicidal behavior in a study of 137 patients and 71 controls. Id. Measures of kynurenine metabolites can be explored as biomarkers of suicide risk, and that ACMSD is a potential therapeutic target in suicidal behavior. Id. It is believed that the presence of the C allele of the SNP rs2121337 would be associated with a higher response to L-4-CI-KYN.

L-4-CI-KYN has been synthesized by the methods of U.S. Pat. No. 5,547,991, and Salituro, F. G. et al. J. Med. Chem. 1994, 37, 334-336. And preferred synthesis methods are described in U.S. Pat. No. 9,834,801, and in International Patent Application No. PCT/US2019/07448. L-4-CI-KYN also is available commercially from various sources, including BOC Sciences (Shirley, N.Y., USA), Advanced Technology & Industrial Co., Ltd. (Hong Kong, China), and Cambridge Major Laboratories (Germantown, Wis., USA).

The invention generally relates to treatment of a subject with neurological disorders, including, but not limited to, depression, major depressive disorder, and/or suicidal ideation, by administering L-4-CI-KYN to the subject in a dose based on whether the subject has at least one of a KMO gene variant, wherein the gene variant is a T allele of the SNP rs61825638, a SLC7A5 gene variant, wherein the gene variant is a T allele of the SNP rs28582913, a BDNF gene variant, wherein the gene variant is the common G allele at the location of rs6265, or a ACMSD gene variant, wherein the gene variant is a C allele of rs2121337.

Methods of the invention include detecting at least one of a KMO gene variant, a SLC7A5 gene variant, a BDNF gene variant, and an ACMSD gene variant, in a subject suffering from depression, major depressive disorder, and/or suicidal ideation, the method comprising: obtaining a sample from said subject; and assaying the sample to detect one or more of: (i) a T allele of the SNP r561825638; (ii) a T allele of the SNP r528582913; (iii) the common G allele at the location of rs6265; and (iv) a C allele of the SNP rs2121337. The presence of a T allele of the single nucleotide polymorphism (SNP) rs61825638 in the sample indicates the presence of a KMO gene variant in the subject. The presence of a T allele of the SNP rs28582913 indicates the presence of a SLC7A5 neutral amino acid transporter gene variant in the subject. The presence of the A allele of SNP rs6265 indicates the presence of a BDNF gene variant in the subject. The presence of the C allele of the SNP rs2121337 indicates the presence of an ACMSD gene variant in the subject.

Methods of the invention also include predicting potential responsiveness by detecting at least one of a KMO gene variant, a SLC7A5 gene variant, a BDNF gene variant, and a ACMSD gene variant, in a subject suffering from depression, major depressive disorder, and/or suicidal ideation, the method comprising: obtaining a sample from said subject; and assaying the sample to detect one or more of: (i) a T allele of the single nucleotide polymorphism (SNP) r561825638; (ii) a T allele of the SNP r528582913; (iii) the common G allele at the location of rs6265; and (iv) a C allele of the SNP rs2121337.

The sample can be a genomic DNA sample, RNA sample, cDNA sample, or protein sample obtained from a tissue or bodily fluid of the subject, e.g., blood or saliva. The assaying step can be performed by, for example, DNA sequencing, restriction enzyme digest, polymerase chain reaction (PCR), hybridization, real-time PCR, reverse transcriptase PCR, or ligase chain reaction, and an immunoassay. Several methods for detecting SNPs are available in the literature. See e.g., Kwok, et al., “Detection of Single Nucleotide Polymorphisms.” Curr. Issues Mol. Biol. (2003) 5: 43-60; Mast, A. and M. de Arruda (2006). “Invader assay for single-nucleotide polymorphism genotyping and gene copy number evaluation.” Methods Mol. Biol. 335: 173-186; Zascavage, R. R., et al. (2013). “Deep-Sequencing Technologies and Potential Applications in Forensic DNA Testing.” Forensic Sci. Rev. 25(1-2): 79-105; Ishikawa, T., et al. (2010). “Emerging new technologies in Pharmacogenomics: rapid SNP detection, molecular dynamic simulation, and QSAR analysis methods to validate clinically important genetic variants of human ABC Transporter ABCB1 (P-gp/MDR1).” Pharmacol. Ther. 126(1): 69-81; Chang, K., et al. (2015); “Novel biosensing methodologies for improving the detection of single nucleotide polymorphism.” Biosens. Bioelectron. 66: 297-307; Benitez, J. A., et al. (2017). “Revealing allele-specific gene expression by single-cell transcriptomics.” Int. J. Biochem. Cell Biol. 90: 155-160.

Symptoms of depression include low mood, diminished interest in activities, psychomotor slowing or agitation, changes in appetite, poor concentration or indecisiveness, excessive guilt or feelings of worthlessness, and suicidal ideations may occur in the context of depressive disorders, bipolar disorders, mood disorders due to a general medical condition, substance-induced mood disorders, other unspecified mood disorders, and also may be present in association with a range of other psychiatric disorders, including but not limited to psychotic disorders, cognitive disorders, eating disorders, anxiety disorders and personality disorders. The longitudinal course of the disorder, the history, and type of symptoms, and etiologic factors help distinguish the various forms of mood disorders from each other.

Major Depressive Disorder is defined as the presence of one of more major depressive episodes that are not better accounted for psychotic disorder or bipolar disorder. A major depressive episode is characterized by meeting five or more of the following criteria during the same 2 week period which represent a change in functioning and include at least depressed/sad mood or loss of interest and pleasure, indifference or apathy, or irritability and is usually associated with a change in a number of neurovegetative functions, including sleep patterns, appetite and body weight, motor agitation or retardation, fatigue, impairment in concentration and decision making, feelings of shame or guilt, and thoughts of death or dying (Harrison's Principles of Internal Medicine, 2000). Symptoms of a depressive episode include depressed mood; markedly diminished interest or pleasure in all, or almost all, activities most of the day; weight loss when not dieting or weight gain, or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness, nearly every day; recurrent thoughts of death, recurrent suicidal ideation without a specific plan, or a suicidal attempt or a specific plan for committing suicidal. Further, the symptoms cause clinically significant distress or impairment in social, occupational, or other important areas of functioning. (Diagnostic and Statistical Manual of Mental Disorders, 4^{th} Edition, American Psychiatric Association, 1994).

Suicidal ideation which may include, for example, suicidal thoughts, may also include other related signs and symptoms. Some symptoms or co-morbid conditions may include unintentional weight loss, feeling helpless, feeling alone, excessive fatigue, low self-esteem, presence of consistent mania, excessively talkative, intent on previously dormant goals, feel like one's mind is racing. The onset of symptoms like these with an inability to get rid of or cope with their effects, a possible form of psychological inflexibility, is one possible trait associated with suicidal ideation. They may also cause psychological distress, which is another symptom associated with suicidal ideation. Symptoms like these related with psychological inflexibility, recurring patterns, or psychological distress may in some cases lead to the onset of suicidal ideation. Other possible symptoms and warning signs include: hopelessness, anhedonia, insomnia, depression, severe anxiety, angst, impaired concentration, psychomotor agitation, panic attack and severe remorse.

As will be known to persons skilled in the art, assessment scales used in the evaluation of suicidal ideation include the Beck Scale for Suicidal Ideation (BSS), Columbia Suicidal Severity Rating Scale (C-SSRS), Suicidal Ideation and Behavioral Assessment Tool (SIBAT) and The Kessler Psychological Distress Scale (K10, which test does not measure suicidal ideation directly, but there may be value in its administration as an early identifier of suicidal ideation. High scores of psychological distress are also, in some cases associated with suicidal ideation.

There are also several psychiatric disorders that appear to be comorbid with suicidal ideation or considerably increase the risk of suicidal ideation. The following disorders have been shown to be the strongest predictors of suicidal ideation/disorders in which risk is increased to the greatest extent: major depressive disorder (MDD), dysthymia, bipolar disorder. The main treatments for suicidality and/or suicidal ideation include: hospitalization, out-patient treatment, and medication. Hospitalization allows the subject to be in a secure, supervised environment to prevent their suicidal ideation from turning into suicidal attempts. In most cases, individuals have the freedom to choose which treatment they see fit for themselves. However, there are several circumstances in which individuals can be hospitalized involuntarily, per state law including circumstances where an individual poses danger to self or others and where an individual is unable to care for one's self.

Methods of the invention include treating depression, major depressive disorder, and/or suicidal ideation in a subject, the method comprising: a) obtaining a sample from said subject; b) assaying the sample to detect at least one of a KMO gene variant, wherein the gene variant is a T allele of the SNP rs61825638, a SLC7A5 neutral amino acid transporter gene variant, wherein the gene variant is a T allele of the SNP rs28582913, a BDNF gene variant, wherein the gene variant is the common G allele at the location of rs6265, or a ACMSD gene variant, wherein the gene variant is a C allele of r52121337; and c) administering a therapeutically effective amount of L-4-CI-KYN to the subject to treat the depression, major depressive disorder, or suicidal ideation in the subject, with a dose that is based on the presence of at least one of the detected variants in the sample.

Methods of the invention may be performed on a subject for a wide variety of neurological disorders that are treatable with L-4-CI-KYN where this drug requires transport across the blood brain barrier before it is metabolized to 7-CI-kynureninic acid in the central nervous system. The subject may be a human or mammal suffering, for example, from depression or MDD. Other indications that respond to 7-CI-kynureninic acid metabolized in the CNS from L-4-CI-KYN after its transport into the brain, include, by way of additional examples, various types of neuropathic pain, including hyperalgesia, and neuropathic pain associated with diabetes, chemotherapy or amputation. The common feature of such treatment is the administration of a therapeutically effective amount of L-4-CI-KYN depending on the genotype of the subject according to the methods of the present invention. Other indications include obsessive compulsive disorder (OCD), tinnitus, autoimmune anti-NMDA receptor encephalitis, anxiety, dysthymic disorder (or dysthymia), persistent depressive disorders, atypical depression, bipolar depression or manic depression, seasonal affective disorder (SAD), psychotic depression and postpartum depression, psychotic depression, premenstrual syndrome, premenstrual dysphoric disorder, anxiety, mood disorder, depressions caused by chronic medical conditions such as cancer or chronic pain, chemotherapy, chronic stress, post-traumatic stress disorders, risk of suicidal ideation.

Such therapies optionally include the co-administration of L-4-CI-KYN along with another antidepressant or mood elevating therapy including cognitive therapy and psychotherapy. Methods of the invention also include treatment of various types of neurological disorders, neuropathies (both central and peripheral), pain and dysfunction such as those caused by: (a) injury and drug toxicities such as result from chemotherapy and anti-viral drugs; (b) diseases and neurodegenerative disorders such as diabetes, cancer, viral infection, Multiple Sclerosis, spondylitis, polyneuritis, surgery, amputation, epilepsy, convulsions, Parkinson's disease, Huntington's disease, and Alzheimer's disease and those diseases and conditions involving overactive glutamatergic transmission which can be ameliorated by down-regulating N-methyl-D-aspartate receptor signaling; and (c) imbalances in neurotransmitters, receptors and signaling pathways associated with depression and other psychiatric disorders which also can be ameliorated by down-regulating N-methyl-D-aspartate receptor signaling.

In some methods of treatment according to the invention, L-4-CI-KYN is administered as a pharmaceutical formulation. Preferably, pharmaceutical compositions of L-4-CI-KYN comprise a unit dose of L-4-CI-KYN that is formulated for oral administration, together with pharmaceutically acceptable carriers and excipients. In other embodiments, L-4-CI-KYN is administered as a suppository. “Pharmaceutical unit dose,” “unit dose,” or “unit dose form” means a single dose of L-4-CI-KYN, which is administered to a subject. In some embodiments, the unit dose can be readily handled and packaged, remaining as a physically and chemically stable unit dose. For example, in a particular embodiment, an oral unit dose may be a single tablet, while in another embodiment, an oral unit dose may be more than one tablet.

In some methods of treatment according to the invention, the L-4-CI-KYN is administered as a unit dose, optionally with a pharmaceutically acceptable carrier or excipient, where the amount of L-4-CI-KYN in the unit dose ranges from about 50 mg to about 1,800 mg. For example, the amount of L-4-CI-KYN in the unit dose is preferably from about 260 mg to about 1,540 mg, more preferably either about 260 mg to about 460 mg, about 310 mg to about 410 mg, about 460 mg to about 980 mg, about 980 mg to about 1,180 mg, about 1,030 mg to about 1,130 mg, about 1,340 to about 1,540 mg, about 1,390 mg to about 1,490 mg and most preferably about 360, 720, 1,080, or 1,440 mg. In some methods of treatment according to the invention, a unit dose of a therapeutically effective amount of L-4-CI-KYN that is formulated for oral administration, together with pharmaceutically acceptable carriers and excipients.

It is contemplated that the exact dosages of L-4-CI-KYN to be administered within the ranges described for the present invention are to be safe and effective, and that they produce plasma levels of 7-chlorokynurenic acid resulting from the administration of L-4-CI-KYN as described in U.S. Pat. No. 9,993,453. Thus, plasma ranges of 7-chlorokynurenic acid from about 15 ng/mL to about 65 ng/m, from about 65 ng/mL to about 300 ng/mL and from about 300 ng/mL to about 550 ng/mL are expressly contemplated. It is also contemplated that the unit dose formulation may be administered one or more times per day, in order to extend the time period in which L-4-CI-KYN levels are elevated to a therapeutically effective amount of 7-chlorokynurenic acid.

It is contemplated that the dosing regimens for the compositions of the present invention are therapeutically effective. While a daily dosing regimen is contemplated, this would preferably be from about 5 to about 30 days, including shorter and longer dosing regimens as determined by a subject's physician. In particular, dosing regimens of about 7 to about 24 days, and about 12 to about 16 days are expressly contemplated. A daily dosing regimen can include administration of one or more unit doses per day. In a preferred embodiment, the daily dose does not exceed 2,900 mg/day.

“Therapeutically effective” means that the amount of L-4-CI-KYN administered and converted to 7-chlorokynurenic acid acts to down-regulate NMDA-R mediated signal transmission or an imbalance in neurotransmitters that is sufficient to produce a clinical improvement in neurological function, such as, for example, a decrease in neuropathic pain, or an increase in feelings of well-being or reduction in depressive mood or feelings.

The invention also relates to methods of predicting the potential responsiveness to treatment of a subject based on a subject's genotype. A method of the invention for predicting the potential responsiveness to treatment with L-4-CI-KYN administered to a subject suffering from depression, major depressive disorder, and/or suicidal ideation with treatment, includes: a) obtaining a sample from a subject suffering from depression, major depressive disorder, and/or suicidal ideation; b) assaying the sample to detect at least one of a KMO gene variant, wherein the gene variant is a KMO gene variant, wherein the gene variant is a T allele of the SNP rs61825638, a SLC7A5 neutral amino acid transporter gene variant, wherein the gene variant is a T allele of the SNP rs28582913, a BDNF gene variant, wherein the gene variant is the common G allele at the location of rs6265, or an ACMSD gene variant, wherein the gene variant is a C allele of r52121337; and wherein the absence or presence of at least one of the gene variants is indicative of the potential responsiveness to treatment.

The method also predicts the potential level of responsiveness to treatment based on the subject's genotype. For example, the presence of at least one of the gene variants indicates that a subject will be a relatively higher responder, i.e., requires less drug, or its administration for a shorter duration, as compared to a subject with neither gene variant. The presence of multiple gene variants, for example, a T allele of the SNP rs61825638, a T allele of the SNP rs28582913, and a C allele of rs2121337 indicates that a subject will be a relatively very high responder, i.e., requiring even less drug, or for an even shorter duration, or both, compared to a subject with just one gene variant. On the other hand, the presence of the common G allele at the location of rs6265 indicates that the subject is a low responder, for example, requiring more drug, or for a longer duration, compared to a subject without that gene variant.

### EXAMPLES

### Example 1: Detection of KMO Gene Variant

DNA is extracted from the subject's blood sample. Detection of the gene variant is performed via the T allele-specific primer extension method. The PCR product containing the polymorphic site serves as template and the 3′-end of the primer extension probe consists of the allelic base. The primer is extended only if the 3′-base complements the allele present in the target DNA. Monitoring the primer extension event therefore allows one to infer the allele(s) found in the DNA sample (Pastinen et al. (2000) Genome Res. 10: 1031-1042.).

### Example 2: Detection of SLC7A5 Gene Variant

DNA is extracted from the subject's blood sample. Detection of the gene variant is performed via PCR using the T allele-specific method described above using primers specific for the variant.

### Example 3: Detection of BDNF Gene Variant

DNA is extracted from the subject's blood sample. Detection of the gene variant is performed via PCR using the A allele-specific method described above using primers specific for the variant.

### Example 4: Detection of the ACMSD Gene Variant

DNA is extracted from the subject's blood sample. Detection of the gene variant is performed via PCR using the C allele-specific method described above using primers specific for the variant.

### Example 5: Treatment of Depression

Without being held to a particular mechanism of action, it is believed that because 7-chlorokynurenic targets, that is to say it blocks or antagonizes, the glycine co-agonist site of the NMDA receptor, therapeutic administration of 4-CI-KYN may avoid causing the potential psychotomimetic side effects that occur with ketamine, as determined by the drug discrimination, conditioned place preference, and pre-pulse inhibition tests, while maintaining efficacy. Administration of 4-CI-KYN may then result in the “glutamate surge” resulting in an AMPA receptor-dependent synaptogenesis that has been associated with the rapid acting antidepressant effects of ketamine.

It is also contemplated that L-4-CI-KYN is effective to treat suicidal subjects, or subjects undergoing seizures in acute emergency situations such as in a hospital emergency room. For this use of the present invention, administration of L-4-CI-KYN as an injectable or suppository formulation is preferred for those subjects that are not able to take oral administration. Appropriate formulations are known to persons skilled in the art and are described above.

The preferred dose range for the treatment of depression is from about 20 mg/day up to about 2,900 mg/day, preferably from 150 mg/day to 1000 mg/day, more preferably from about 300 mg/day to about 1,500 mg/day and even more preferably from about 700 mg/day to about 1,200 mg/day. Within these preferred dose ranges, 340 mg/day, 1,080 mg/day and 1,440 mg/day also are preferred.

A mood enhancing or anti-depressive activity of L-4-CI-KYN is observed for a higher number of subjects, or for the same number of patients but at a lower dose, with at least one of a KMO gene variant, wherein the gene variant is a T allele of the SNP rs61825638, a SLC7A5 neutral amino acid transporter gene variant, wherein the gene variant is a T allele of the SNP rs28582913, and an ACMSD gene variant, wherein the gene variant is a C allele of the SNP rs2121337. A mood enhancing or anti-depressive activity of L-4-CI-KYN is observed for a higher number of subjects, or for the same number of patients but at a lower dose, without a BDNF variant, wherein the BDNF variant is the common G allele at the location of rs6265. In subjects with the BDNF variant, the A allele of the SNP rs6265 a higher dose of the L-4-CI-KYN results in the mood-enhancing or anti-depressive response. Subjects affirmatively report feelings of well-being. This is consistent with reports that the glutamatergic system contributes to the pathophysiology of depression and that stress can induce changes in NMDA receptors. See, for example, Calabrese, et al., PloS one vol. 7,5 (2012): e37916.

### Example 6: Treatment of MDD

Twenty-five subjects, both male and female, ages 18 to 65, with a diagnosis of MDD are treated with either L-4-CI-KYN (1,080 or 1,440 mg/day given orally) for 2 weeks, similar in design to similar studies. See e.g., Ibrahim et al. J Clin Psychopharmacol. 2012 August; 32(4): 551-557; Zarate et al. Biol Psychiatry. 2013 Aug. 15; 74(4):257-64; Zarate et al. Arch Gen Psychiatry. 2006 August; 63(8):856-64; Zarate et al. 2005 Biological psychiatry 57 (4), 430-432. The subjects are pre-screened to be positive for at least one of the following variant SNPs, the T allele of SNP rs61825638, the T allele of SNP rs28582913, or the C allele of rs2121337, or positive for the G allele of rs6265. In addition, a control group of twenty-five subjects is pre-screened as lacking any of: the T allele of SNP rs61825638, the T allele of SNP rs28582913, or the C allele of rs2121337, or lacking the G allele of SNP rs6265. Improvement in overall depressive symptomatology is shown by a significant decrease in either or both of the Hamilton Depression Rating Scale (HDRS) (Hamilton, M. (1959) J. Med. Psychol. 32:50-5) and the Montgomery Asberg Depression Rating Scale (MADRS) total scores. Montgomery et al. 1979, Brit. J. Psych., 134:382-389.

As will be understood by persons skilled in the art, additional indicators of therapeutic efficacy for a given subject include a subject achieving remission (HDRS 7) and a therapeutic response (50% reduction from pretreatment baseline in HDRS total score); change from baseline in Hamilton Anxiety Rating Scale (HAM-A), Columbia Suicidal Severity Rating Scale (C-SSRS) total scores (Posner et al. 2011, Am J Psychiatry. 2011; 168:1266-1277), as well as other measures of improved mood or psychological states, for example Beck Depression Inventory (BDI) (Beck et al., 1974, Journal of Consulting and Clinical Psychology, 42(6), 861-865) the Visual Analogue Scale (VAS) (Aitken RC, Proc R Soc Med. 1969; 62(10):989-93), the Brief Psychiatric Rating Scale (BPRS) (Overall et al. 1962, Psych. Rep. 10:799-812) the Clinician Administered Dissociative Scale (CADSS) (Bremner et al. 1998, Journal of Traumatic Stress, 11:125-136) and the Young Mania Rating Scale (YMRS) (Young et al. 1978, British Journal of Psychiatry, 133(5), 429-435).

While certain exemplary embodiments have been described above in detail, it is to be understood that such embodiments are merely illustrative of and not restrictive of the broad invention. It should be recognized that the teachings of the invention apply to a wide variety of compositions and devices produced from the formulations and compositions described. Persons of skill in the art will recognize that various modifications may be made to the embodiments of the invention described above, without departing from its broad inventive scope. Thus, it will be understood that the invention is not limited to the embodiments or arrangements disclosed, but is rather intended to cover any changes, adaptations or modifications which are within the scope and spirit of the invention as defined by the appended claims. All documents discussed or cited in this specification are incorporated by reference in their entirety.

What is claimed:

1. A method of predicting responsiveness to treatment with L-4-CI-KYN by detecting at least one of a kynurenine 3-monooxygenase (KMO) gene variant, a SLC7A5 neutral amino acid transporter gene variant, a brain derived neurotrophic factor (BDNF) gene variant, and an aminocarboxymuconate semialdehyde decarboxylase (ACMSD) gene variant, in a subject suffering from depression, major depressive disorder, and/or suicidal ideation, the method comprising:
obtaining a sample from said subject; and
assaying the sample to detect one or more of:
(i) a T allele of the single nucleotide polymorphism (SNP) r561825638;
(ii) a T allele of the SNP r528582913;
(iii) G allele of the SNP rs6265; and
(iv) a C allele of the SNP rs2121337.

2. The method of claim 1, wherein the sample is a genomic DNA sample.

3. The method of claim 1, wherein the sample is an RNA sample.

4. The method of claim 1, wherein the sample is obtained from the blood or saliva of the subject.

5. The method of claim 1, wherein the assaying step is performed by DNA sequencing, restriction enzyme digest, polymerase chain reaction (PCR), hybridization, real-time PCR, reverse transcriptase PCR, or ligase chain reaction.

6. A method for the treatment of depression, major depressive disorder, and/or suicidal ideation in a subject, the method comprising:
a) obtaining a sample from said subject;
b) assaying the sample to detect at least one of
a KMO gene variant, wherein the gene variant is a T allele of the single nucleotide polymorphism (SNP) rs61825638,
a SLC7A5 neutral amino acid transporter gene variant, wherein the gene variant is a T allele of the SNP rs28582913
a BDNF gene variant, wherein the gene variant is a G allele at the location of rs6265, and
an ACMSD gene variant, wherein the gene variant is a C allele of the SNP r52121337; and
c) administering a therapeutically effective amount of L-4-CI-KYN to the subject to treat the depression, major depressive disorder, or suicidal ideation in the subject, based on the presence of at least one of the detected variants in the sample.

7. The method of claim 6, wherein the method is for the treatment of depression.

8. The method of claim 7, wherein the depression is treatment resistant depression.

9. The method of claim 6, wherein the method is for the treatment of major depressive disorder.

10. The method of claim 9, wherein the major depressive disorder is treatment resistant major depressive disorder.

11. The method of claim 10, wherein the method is for the treatment of suicidal ideation.

12. The method of claim 10, wherein the subject is a suicidal subject.

13. A method of predicting a subject's potential responsiveness to treatment with L-4-CI-KYN administered to a subject suffering from depression, major depressive disorder, and/or suicidal ideation with treatment, the method comprising:
a) obtaining a sample from a subject suffering from depression, major depressive disorder, and/or suicidal ideation;
b) assaying the sample to detect at least one of
a KMO gene variant, wherein the gene variant is a T allele of the single nucleotide polymorphism (SNP) rs61825638,
a SLC7A5 neutral amino acid transporter gene variant, wherein the gene variant is a T allele of the SNP rs28582913
a BDNF gene variant, wherein the gene variant is a G allele of SNP rs6265, and
an ACMSD gene variant, wherein the gene variant is a C allele of the SNP r52121337; and
wherein the absence or presence of at least one of the gene variants is indicative of the subject's responsiveness to treatment.
